(19) United States
(12) Patent Application Publication
(10) Pub. No.: US 2026/0318886 A1
(43) Pub. Date: Oct. 15, 2026

(54) COPPER CORROSION TEST METHOD

(71) Applicant: SHELL USA, INC., Houston, TX (US)

(72) Inventors: Gwenaelle Sophie Olivia PHILIBERT, Houston, TX (US); Jiayi Lu, Houston, TX (US); Christopher Claus DOBROWOLSKI, Hamburg (DE)

(21) Appl. No.: 19/104,503

(22) PCT Filed: Sep. 20, 2023
(86) PCT No.: PCT/EP2023/075928

§ 371 Date: Feb. 18, 2025

Related U.S. Application Data
(60) Provisional application No. 63/408,960, filed on Sep. 22, 2022.

(51) Int. Cl. G01N 30/72; G01N 1/44; G01N 30/02; G01N 33/28

(52) U.S. Cl. CPC G01N 30/72; G01N 1/44; G01N 33/2888; G01N 2030/027

(57) ABSTRACT

This invention provides a method for assessing the copper corrosion potential of a lubricant fluid comprising the steps of: a) placing a sample of said lubricant fluid in a vial, wherein said sample partially fills said vial, such that a space exists in the vial above the sample suitable for sampling vapour phase materials in said space; b) sealing the vial; c) heating the sample in said sealed vial for greater than 12 hours at a temperature of at least 80° C.; and d) analysing the vapour phase portion using headspace GC-MS with full scan and selected ion monitoring.

### FIELD OF THE INVENTION

This invention relates to a test method for readily evaluating the vapour phase copper corrosion potential of a lubricant fluid.

### BACKGROUND OF THE INVENTION

As the energy transition progresses, there is an increased demand for transportation using, at least in part, electric powered motors or ‘e-motors’. Copper is an important component in e-motors due to its electrical conductivity, heat transfer ability, ductility and cost. Within an e-motor, copper windings are typically protected by a coating. However, at connection points and due to coating degradation, copper may become exposed.

Typically, in current e-motors, there are two usual configurations: a dry e-motor, in which the lubricant fluid used in the transmission is kept separate from the e-motor; and a wet e-motor, in which a lubricant fluid is used to lubricate and cool the e-motor in addition to lubricating the gearbox. In a wet e-motor, there is an increased risk of the fluid coming into contact with the copper winding of the e-motor, either in the liquid phase or vapor phase or both.

The development of new lubricant fluids suitable for use in e-motors has been the focus of much research. It is important for these fluids to exhibit low levels of copper corrosion. Copper corrosion is thought to be caused by a number of factors. The presence of sulfur and nitrogen compounds in the base oil or derived from certain additives used in the lubricant fluid are known to contribute to corrosion as they degrade at higher temperatures. The formulation of new lubricant fluids for use in e-motors requires extensive, time-consuming testing of potential candidates to determine their copper corrosion potential.

Testing for copper corrosion in hydrocarbon products has historically been based on ASTM D130, in which a strip of copper is immersed in fluid, e.g., an oil, and heated for a number of hours. A visual rating of the used copper strip on a scale from 1a to 4c is then given, depending on the level of discolouration. This method provides a clear differentiation between high levels of copper corrosion and low levels of copper corrosion but cannot provide the precision needed for comparison between low levels of corrosivity.

ASTM D130 is a relatively old test and was not developed to test lubricants for use in an e-motor. A number of attempts have been made to improve the test for more modern requirements. For example, to improve the sensitivity of the test, it is possible to extend it by analysing the oil sample in which the copper strip has been immersed for copper concentration at the end of the test with inductively coupled plasma (ICP), in order to quantify copper losses. It is also possible to modify the test by suspending the copper strip above the fluid sample, or by only partly immersing the copper strip, to assess the vapour phase copper corrosion potential of a fluid.

A so-called “energized corrosion and conductive deposit” test has been developed to test the compatibility of a fluid with electronic components under energized conditions. This conductive deposit test, described in G. Müller; J. Bucci; G. Mueller; R. Pelz; T. Newcomb; A. Gangopadhyay, “Conductive Layer Deposits and the Development of an Effective Bench Test for Electric Vehicle Drivetrains,” in SAE International; 2021, uses a circuit board, half of which is immersed in the fluid, the other half exposed to the vapour. The fluid is typically heated at 150° C. and a 5V DC power is applied to the board. Resistance is monitored over time both in the liquid and vapor phase. Pass/fail thresholds are still being evaluated, but it is currently considered that failure can be determined when resistance decreases due to a shorting deposit within 1000 hours.

A further copper corrosion test is described in G. Hunt, M. Gahagan and M. Peplow, Lubrication science, vol. 29, no. 4, pp. 279-290, 2017. In this test, two bare wires are placed in a test vessel. One is immersed in the test fluid while the other is suspended above the fluid. A direct current (DC) of 1 mA is applied. The test fluid is held at the desired temperature and the suspended wire will be exposed to the vapor of the test fluid. The wire resistance is monitored over time. As each wire corrodes, some conducting copper metal is lost and so the electrical resistance will increase. Corrosion can be detected as the change in resistance measured in the circuit due to decrease in conduction cross sectional area. However, this method is difficult to set-up and requires a fresh wire to be installed for each sample.

There remains a desire to test simply and rapidly the vapour phase copper corrosion potential of formulated lubricant fluids. Further, it would be desirable if the testing method could provide insights into the mechanism of copper corrosion being caused by any tested fluid to help with the further development of improved fluid.

### SUMMARY OF THE INVENTION

The present invention provides a method for assessing the copper corrosion potential of a lubricant fluid comprising the steps of:
a) placing a sample of said lubricant fluid in a vial, wherein said sample partially fills said vial, such that a space exists in the vial above the sample suitable for sampling vapour phase materials in said space;
b) sealing the vial;
c) heating the sample in said sealed vial for greater than 12 hours at a temperature of at least 80° C.; and
d) analysing the vapour phase portion using headspace GC-MS with full scan and selected ion monitoring.

### DETAILED DESCRIPTION OF THE INVENTION

The present inventors have found that an accurate assessment of the amount of copper corrosion potential of a lubricant fluid can be readily assessed using Gas-chromatography coupled with mass spectrometry (GC-MS) analysis of the vapour phase above a sample of said lubricant fluid after said sample has been subjected to a pre-treatment comprising heating the sealed sample for greater than 12 hours. In particular, the GC-MS may be used to identify the presence of species including one or more of hydrogen sulfide (H₂S), ammonia (NH₃), carbonyl disulfide (CS₂) and carbonyl sulfide (COS). However, other corrosive species may be identified in both the full scan and by selecting the correct ions for monitoring.

The inventive method provides a simple and quick method to assess the vapour phase copper corrosion potential of a lubricant fluid. It has been demonstrated to produce results that correlate well with industry known methods that are considerably more complex and time-consuming. The inventive method may be used, for example, to provide a rapid assessment of a large number of candidate lubricant fluid samples.

In the present invention, a sample of the lubricant fluid is placed in a vial, such that said sample only partially fills that vial and a space exists above the liquid sample. Typically, the sample will fill in the range of from 5 to 20%, preferably less than 20% of the volume of the vial. A larger sample volume increases the chance of leakage and sample loss during the test due to the higher vapour pressure created.

Sealing of the vial is suitably carried out by using a cap, for example, an aluminium cap, incorporating a septum of a pierceable material, such as one or more of silicone, rubber and PTFE.

Headspace GC-MS is a known method. The term “headspace” refers to the fact that the sample is taken from the space above a liquid sample in a vial. In a typical headspace GC-MS method, the sample is sealed in a vial and then heated for a short period of time, for example one hour, to allow for the vapour phase and the liquid sample to equilibrate. An aliquot of the vapor phase is then taken via a needle inserted into the vial and passed by heated transfer line to a gas chromatograph (GC), where species are separated before being identified in the mass spectrometer (MS).

In the present invention, the sample of lubricating fluid is subjected to a pre-treatment in which the sealed sample is heated at a temperature of at least 80° C. for at least 12 hours. Preferably, the temperature is at least 140° C., more preferably at least 170° C. Suitably the pre-treatment step is carried out at a temperature not exceeding 180° C. The pre-treatment step is preferably at least 24 hours, more preferably at least 48 hours. It will be readily understood that a pre-treatment step may be carried out at a higher temperature for less time or a lower temperature for a longer period of time. For example, a suitable pre-treatment step may take place at 120° C. for 72 hours or at 170° C. for 24 hours.

After the pre-treatment step, a portion of the vapour phase above the liquid lubricant fluid sample is removed from the vial and injected into a GC-MS. The GC-MS separates the species in the injected vapour phase portion and analyses them.

Suitable GC-MS conditions will be readily determined by the skilled person.

The use of selected ion monitoring in the present invention allows the assessment of target molecules known for their contribution to copper corrosion. Selected ion monitoring (SIM) is a scanning mode in mass spectrometry in which a single mass to charge ratio is selected to be detected instead of the full range. This scanning mode allows improved sensitivity and removes interference from co-eluting material allowing accurate assessment of the species of interest. Of particular interest in the present invention are ammonia (mass to charge ratio m/z=17), H₂S (mass to charge ratio m/z=34), carbonyl disulfide (mass to charge ration m/z=76) and COS (mass to charge ratio m/z=60). However, the present invention is easily applicable to any selected ion relating to an actual or suspected corrosive species.

The area under the peak on the chromatogram is directly proportional to the concentration of the analyte of interest. The detector can be calibrated using standard compound to establish the response factor between the peak area and the analyte concentration. Alternatively, the peak area of a corrosive specie in a lubricant fluid can be compared to the same measurement taken for a standard lubricant fluid. Typically, the standard lubricant fluid is a known lubricant fluid with a known copper corrosion potential.

The invention will now be further illustrated by reference to the following non-limiting examples.

### EXAMPLES

### Examples 1 to 12

To demonstrate the effectiveness of the inventive method, a number of fluids suitable as lubricant fluids for a wet e-motor were prepared and tested using the method. Each fluid contained GTL base oil and at least one additive package.

In each example, a sample of the fluid was placed in a headspace vial and pre-heated either at 140° C. for 48 hours or at 170° C. for 24 hours.

A headspace sample was taken and analysed in a GC-MS. Conditions: injection duration 1 minute; oven 45° C. for 8 minutes, 20° C./min ramping to 120° C. for 20 minutes; PoraBOND Q column (50 m×0.53 mm×10 μm); inlet 180° C.; split 5:1; helium as a carrier gas.

Selected ion monitoring was used to assess specific vapour phase corrosives. Four potential corrosive species were selected to be assessed using selected ion monitoring. These were ammonia, carbonyl sulfide, carbon disulfide and hydrogen sulfide.

The formulation indicated as Example 1, which was a known fluid, with known copper corrosion potential, was taken as a standard and the area under the GC trace was measured for each of the potential corrosive species. This area was set as 100 for each corrosive species. All other measurements were quantified in comparison to this standard. The results for these Examples are shown in Table 1.

TABLE 1

| | Pre-heating at 140° C./ | | | | Preheating at 170° C./ | | |
|---|---|---|---|---|---|---|---|
| | 48 hours | | | | 24 hours | | |
| Example | NH₃ | COS | H₂S | CS2 | COS | H₂S | CS2 |
| 1 | 100 | 100 | 100 | 100 | 652 | 1702 | 110 |
| 2 | 63 | 256 | 0 | | 929 | 394 | |
| 3 | 125 | 93 | 14 | | 696 | 1270 | |
| 4 | 63 | 118 | 100 | | — | — | |
| 5 | 49 | 67 | 100 | | — | — | |
| 6 | 11 | 167 | 100 | | — | — | |
| 7 | 76 | 137 | 100 | | — | — | |
| 8 | 211 | 11 | 3000 | | — | — | |
| 9 | 104 | 0 | 0 | | — | — | |
| 10 | 45 | 386 | 0 | 210 | 1634 | 404 | 195 |
| 11 | 70 | 28 | 3232 | | 83 | 8420 | |
| 12 | 103 | 22 | 3750 | | 128 | 20427 | |

The effect of changes in formulation, including addition of vapour phase corrosion inhibitors (Examples 2 and 3) and changes in additive package (Examples 4 to 9), on the production of vapour phase corrosive species are clearly shown to be easily identifiable using the method of the present invention. This allows quick and simple assessment of likely vapour phase copper corrosion of a formulated lubricant fluid.

The results from the method of the present invention were then compared to results for some of the same samples using known and more complex copper corrosion test methods.

The samples in Table 2 were each assessed using a conductive deposit test according to G. Müller; J. Bucci; G. Mueller; R. Pelz; T. Newcomb; A. Gangopadhyay, “Conductive Layer Deposits and the Development of an Effective Bench Test for Electric Vehicle Drivetrains,” in SAE International; 2021. The oil sample used in the conductive deposit test was then assessed using inductively coupled plasma (ICP) to assess the level of copper dissolved in the used oil as well as the sulfur loss from the fresh oil at the end of the CDT. The results from these tests are shown in Table 2.

| | | | | | Conductive Deposit Test | | | | | | |
|---|---|---|---|---|---|---|---|---|---|---|---|
| | | | | | | | | | ICP results on Oil | | |
| | | | | | | | | | Samples | | |
| | | | | | Resistance Testing | | | | | S in | S loss |
| | GC-MS data from Inventive | | | | Time to 50000 | | Resistance end | | | fresh | end of |
| | Method | | | | ohms (h) | | of test (ohms) | | Dissolved | oil | test |
| | NH₃ | COS | H₂S | CS₂ | oil | vapour | oil | Vapour | Cu (ppm) | (ppm) | (%) |
| 1 | 100 | 100 | 100 | 100 | 100 | 100 | 100 | 9 | 577 | 1003 | 77 |
| 5 | 49 | 67 | 100 | | 100 | 100 | 10 | 10 | 583 | 1002 | 82 |
| 6 | 11 | 167 | 100 | | 100 | 100 | 1000 | 1000 | 881 | 1014 | 83 |
| 8 | 211 | 11 | 3000 | | 1000 | 1000 | 100000 | 100000 | 1060 | 54 | 80 |
| 9 | 104 | 0 | 0 | | 1000 | 1000 | 100000 | 100000 | 306 | 29 | 0 |
| 10 | 45 | 386 | 0 | 200 | 100 | 159 | 1000 | 50 | 596 | 1172 | 62 |
| 11 | 70 | 28 | 3232 | | 700 | 300 | 100 | 1000 | 399 | 620 | 66 |
| 12 | 103 | 22 | 3750 | | 250 | 200 | 100 | 100 | 1043 | 853 | 46 |

1. A method for assessing the copper corrosion potential of a lubricant fluid comprising the steps of:
a) placing a sample of said lubricant fluid in a vial, wherein said sample partially fills said vial, such that a space exists in the vial above the sample suitable for sampling vapour phase materials in said space;
b) sealing the vial;
c) heating the sample in said sealed vial for greater than 12 hours at a temperature of at least 80° C.; and
d) analysing the vapour phase portion using headspace GC-MS with full scan and selected ion monitoring.

2. A method as claimed in claim 1, wherein selected ion monitoring is used to identify the presence of species including one or more of NH₃, COS, CS₂ and H₂S.

3. A method as claimed in claim 1, also comprising step e) comparing the results obtained in step d) with those obtained applying steps a) to d) to a further lubricant fluid with known copper corrosion properties.

4. A method as claimed in claim 1, wherein in step c) the sample is heated in said sealed vial at a temperature of at least 100° C.

5. A method as claimed in claim 4, wherein in step c) the sample is heated in said sealed vial at a temperature of 140° C. for 48 hours or a temperature of 170° C. for 24 hours.

6. A method as claimed in claim 1, wherein selected ion monitoring is used to assess the presence of one or more of NH₃, COS, CS₂ and H₂S.
